(19) United States
(12) Patent Application Publication
(10) Pub. No.: US 2024/0301036 A1
(43) Pub. Date: Sep. 12, 2024

(54) TREATMENT OF INTESTINAL LUMEN IMMUNOGLOBULIN DEFICIENCY WITH SEMISYNTHETIC POLYCLONAL HUMAN SECRETORY IMMUNOGLOBIN A

(71) Applicant: Michael R. Simon, Ann Arbor, MI (US)

(72) Inventor: Michael R. Simon, Ann Arbor, MI (US)

(21) Appl. No.: 18/595,744

(22) Filed: Mar. 5, 2024

Related U.S. Application Data
(60) Provisional application No. 63/450,465, filed on Mar. 7, 2023.

(51) Int. Cl. C07K 16/06; A61K 9/00; A61K 39/395; A61K 45/06

(52) U.S. Cl. CPC C07K 16/06; A61K 9/0053; A61K 39/39516; A61K 45/06; C07K 2317/35; C07K 2317/94; C07K 2319/31

(57) ABSTRACT

A process is provided for inhibiting symptoms or correction of gastrointestinal antibody deficiency in a subject that includes the oral administration to the subject of a human polyclonal secretory IgA formed by the conjugation of human recombinant secretory component and pooled polyclonal human plasma derived dimeric and polymeric. When administered in a therapeutic quantity, symptoms of intestinal lumen antibody deficiency in the subject are inhibited or prevented.

### RELATED APPLICATIONS

This application claims priority benefit of U.S. Provisional Application Ser. No. 63/450,465 filed 7 Mar. 2024; the contents of which are hereby incorporated by reference.

### GOVERNMENT SUPPORT

This invention was made with government support under 1R44DK130749-01A1 awarded by the National Institutes of Health. The government has certain rights in the invention.

### FIELD OF THE INVENTION

This invention relates to a process for the prophylaxis, treatment, or correction of intestinal lumen antibody deficiency with orally administered human secretory IgA in a subject suffering therefrom or prone thereto, the composition administered in the form of an oral pharmaceutical composition.

### BACKGROUND OF THE INVENTION

Intestinal lumen antibody deficiency is caused by several diseases. The antibody deficiency origin can be either hereditary or acquired. It may occur as a manifestation of the many primary immunodeficiency disorders which are characterized by defective humoral immunity (Agarwal and Cunningham-Rundles 2019). IgA deficiency and common variable immunodeficiency disorder (CVID) are the most common of these conditions. The prevalence of selective IgA deficiency varies from 1:100 to 1:1000 (Agarwal and Cunningham-Rundles 2019). There is a 10 to 20 fold increase in risk for associated gastrointestinal infection and inflammation.

Infection most commonly includes Giardia lamblia and inflammatory disease manifests as nodular lymphoid hyperplasia. The latter regresses after successful treatment of the Giardia infection (Agarwal and Cunningham-Rundles 2019). Both infection and inflammation may also occur in CVID. Gastrointestinal infections include Giardia, Salmonella, Campylobacter and cytomegalovirus (Oksenhendler 2008). Inflammatory changes in the intestinal tissue also include nodular lymphoid hyperplasia, and reduced plasma cells with increased intraepithelial lymphocytes (Khan R et al 2020). The prevalence of intestinal antibody deficiency is 1 in 30,000 (Weifenbach 2020) (Khan R et al 2020).

The etiology of intestinal antibody deficiency has no clear connections to other conditions of enterocolitis, celiac disease, C. difficile infection, food intolerance, and food allergy. The mainstay of medical treatment of CVID is immunoglobulin replacement therapy (AAAAI Guidelines). This treatment includes intravenous infusions of IgG (IVIg) or subcutaneous administration of IgG. However, replacement gastrointestinal polyclonal human secretory IgA has not been considered as a basis for treatment.

The field has remained focused in infusions to the exclusion of contemplation of orally administration of IgA.

Thus, there exists a need for a human polyclonal secretory IgA therapeutic for the treatment of antibody deficiency diseases There also exists a need to provide such a therapeutic in a dosing form well suited for treating an affected infant.

### SUMMARY OF THE INVENTION

A process is provided for inhibiting symptoms or correction of intestinal lumen antibody deficiency, especially selective IgA deficiency and CVID in a subject suffering therefrom or prone thereto that includes the oral administration of polyclonal human semisynthetic secretory IgA to the subject. When administered in a therapeutic quantity based on the subject characteristics and the type of IgA, symptoms of intestinal lumen antibody deficiency in that subject are inhibited. The administered immunoglobulin is readily formed from polyclonal sources. This invention specifies an industrial method for the manufacture of polyclonal human secretory IgA which is not otherwise obtainable in amounts suitable for medicinal use. The IgA is readily administered in a dimeric, or polymeric form that includes recombinant human secretory component (semisynthetic secretory IgA).

### DETAILED DESCRIPTION OF THE PREFERRED EMBODIMENTS

The present invention has utility as a prevention, treatment, or correctio of infections and inflammation resulting from intestinal lumen antibody deficiency through resort to human polyclonal secretory IgA. This is readily prepared by addition of recombinant human secretory component (semisynthetic secretory IgA) as a potential medicament specifically for the treatment of gastrointestinal complications of antibody deficiency diseases. The process of treatment or prevention includes treatment with polyclonal-secretory IgA that is, dimeric or polymeric and polyclonal. Polyclonal dimeric or polymeric IgA is recoverable from the plasma fractionation waste product Cohn fraction III precipitate or equivalent (Simon, et al. 2014). It is also recoverable from the ion exchange plasma fractionation process used to recover other plasma proteins (U.S. Pat. Nos. 9,828,418B2, 10385117B2, 9828418B2).

Because of its resistance to degradation in the gastrointestinal tract, secretory IgA (U.S. Pat. No. 9,932,392B2) can be administered by mouth. Allogeneic immunoglobulins administered directly to the gastrointestinal tract have minimal side effects because they are naturally present in the gastrointestinal tract. Dimeric and polymeric IgA according to the present invention is bound to recombinant human secretory component in order to mimic naturally secreted intestinal secretory IgA which is endogenous to the subject. The administration of the semisynthetic secretory IgA compensates for the absence of naturally secreted secretory IgA in the gastrointestinal tract.

As used herein, a “subject” is defined as a human.

As used herein, “dimeric and polymeric IgA” is defined as a construct that contains two or more IgA monomers plus J chain.

As used herein, “disease correction” is defined as a subject being in a condition in which no further symptoms of the disease appear after having previously been symptomatic.

As the present invention uses an immunoglobulin rather than a metabolic or immunological inhibitor, an effective treatment or preventative is provided which does not disturb the body's metabolism.

The following description of the preferred embodiment(s) is merely exemplary in nature and is in no way intended to limit the invention, its application, or uses.

Secretory IgA molecules are polyclonal and dimeric or polymeric; and are all known to the art, as evidenced for example, by the references incorporated herein.

In particular inventive embodiments, the invention provides a process for medical treatment of humans involving the oral administration of secretory IgA which can be derived from a number of sources. One such source for the IgA is pooled human plasma following Cohn cold ethanol fractionation to produce fraction III precipitate as performed by those of skill in the art of protein separation (Cohn 1946). The IgA byproduct is further purified by adsorption onto jackbean lectin (jacalin) or onto an ion exchange medium in neutral or slightly acidic conditions as performed by those of skill in the art of protein purification (Kabir S, 1998; and U.S. Pat. No. 9,828,418 B2).

A more detailed description of isolation of an IgA component as a byproduct from pooled human plasma or hyperimmune pooled human plasma is as follows. Ethanol fractionation of pooled human plasma is a well-known process to prepare immunoglobulin G. Pooled human plasma is first obtained from licensed plasmapheresis centers in the United States and tested for various pathogens including the HIV virus. The first manufacturing step of most commercial immunoglobulin G preparations involves a modified cold ethanol fractionation according to Cohn to produce Cohn fraction II. In the fractionation process, many infectious viruses are eliminated from the pooled human plasma. Following fractionation, the Cohn fraction II is subjected to adsorption onto an ion exchange medium. This step may selectively reduce the IgA concentration to less than 0.1%. Such a step is important for producing immunoglobulin G for intravenous infusion into humans. The modified cold ethanol fractionation process according to Cohn is a series of fractionations using various levels of ethanol, pH, and temperature to produce a fraction II which is further treated to produce immunoglobulins as described above. In the fractionation method, pooled human plasma is first treated to produce a cryoprecipitate and cryo-supernatant. The cryo-supernatant is subjected to a first ethanol fractionation to yield a supernatant I. Supernatant I is subjected to a second ethanol fractionation to yield fraction II+III. Fraction II+III is subjected to a third ethanol fractionation procedure to yield a supernatant III and Fraction III precipitate.

The fraction III precipitate enriched in IgA is generally discarded as an unwanted byproduct. According to the invention, this unwanted IgA following affinity chromatographic purification is further treated by incubation with immobilized hydrolases to inactivate viruses and vasoactive substances. Such treatment has been proven to eliminate many viruses tested including HIV, Sindbis, and vaccinia. Other antiviral treatments, as known to those skilled in the art, are used in combination and consist of solvent detergent processes, nanofiltration and/or heat inactivation. Usually, three antiviral steps are implemented. Following incubation to remove viruses, the concentration of the active material is adjusted with sterile saline or buffered solutions to ensure a constant amount of active material per milliliter of reconstituted product. Finally, the solution with a constant amount of reconstituted product is sterilized by filtration before use.

The ethanol fractionation process according to Cohn is well known in the art and is described in Cohn et al., 1946 and in more detail in pages 576-602, Kirk-Othmer Encyclopedia of Chemical Technology, Vol. 3, second edition (1963). Alternatively, ion exchange chromatography may be used to obtain the dimeric and polymeric IgA byproduct during the manufacture of intravenous immunoglobulin. From 4% to 22% of plasma IgA is dimeric and polymeric IgA (Delacroix et al., 1981; Delacroix et al., 1983). The resulting dimeric and polymeric IgA is purified. The compositions of the invention contain, in addition to the IgA component, recombinant human secretory component. Human secretory component can be produced by recombinant techniques as described in Crottet et al., 1999. The resulting dimeric IgA is further coupled to recombinant human secretory component. In a preferred embodiment, the coupling is accomplished by forming disulfide bonds under mildly oxidizing conditions (Jones, 1998). Dimeric secretory IgA containing both J chain and secretory component is again purified by ion-exchange and size exclusion chromatography and/or ultrafiltration as described in Lullau et al., 1996; Corthesy, 1997; and Crottet et al., 1999; as performed by those of skill in the art of protein purification. Purified dimeric and polymeric secretory IgA containing recombinant human secretory component is optionally stabilized for example by the addition of human serum albumin to a final concentration of 5%. The presence of the human secretory component in the compositions of the invention leads to doses of immunoglobulin A which are physiologically effective whereas compositions without secretory component is not. Additionally, this invention specifies an industrial method for the manufacture of polyclonal human secretory IgA comprised of recombinant human secretory component plus natural human plasma-derived IgA dimers and higher polymers which would not otherwise be obtainable in quantities sufficient for commercial medicinal use.

In still other embodiments, an IgA is combined with pasteurized human milk or with human milk prepared in a bioreactor (Deng M, 2022).

The secretory IgA antibodies may be administered alone, or with various pharmaceutical adjuvants.

These compositions optionally contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged residence in the intestinal lumen of the IgA can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like.

Solid dosage forms such as tablets, dragees, capsules, pills, and granules can be prepared with coatings and shells, such as enteric coatings and others well known in the art; as detailed, for example in U.S. Pat. Nos. 4,017,647; 4385078; 4518433; and 4556552.

Such solid dosages may contain opacifying agents and can also be of such composition that they release the active compound or compounds in a certain part of the intestinal tract in a delayed manner. Examples of embedding compositions which can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propylene glycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum or other metal hydroxides, bentonite, agar-agar and tragacanth, or mixtures of these substances, and the like.

Since the effect of the IgA antibodies is dependent on their reaching the small intestine, preferred tablets or capsules are enteric coated. Alternatively, the active IgA antibodies can themselves be microencapsulated prior to formulation. Preparation of microcapsules of IgA antibody as well as preparation of enteric coated tablets or capsules can be achieved by conventional methods as detailed above.

It is appreciated that the therapeutic amount of IgA depends on the form thereof, with forms subject to gastrointestinal degradation requiring larger doses. Typical human subject doses of IgA range from about 0.005 mg to 50 grams per day are used and preferably, 1 mg to 40 grams per day. Generally, secretory IgA are each independently effective as a treatment when provided to the patient at about 10 grams per day. Forms of IgA that are prone to gastrointestinal degradation are typically effective in doses increased by at least 80% relative to secretory forms. For example, about 5 grams of secretory IgA could be given to a subject per day in a single dose or in divided doses 3 to 4 times per day. Preferably, multiple doses are administered with meals likely containing food allergens. It is appreciated that a physician can readily adjust the doses of the IgA to be administered based on the subject response to treatment. Many factors are considered in dose adjustments. However, it is to be understood that doses can readily be adjusted to provide appropriate amounts of the IgA antibody.

The invention is further described by reference to the following detailed examples, with exemplary process methodologies described below. These examples are not meant to limit the scope of the invention that has been set forth in the foregoing description. Variations within the concepts of the invention are apparent to those skilled in the art.

The invention is distinguished from the prior art by the derivation of its dimeric and polymeric IgA component from pooled healthy human plasma. It is further distinguished by the conjugation of the dimeric and polymeric IgA components with recombinant human secretory component which is required for its normal activity in the intestines. Importantly, this invention specifies an industrial method for the manufacture of polyclonal semisynthetic human secretory IgA which is not otherwise obtainable in amounts suitable for widespread medicinal use.

### EXAMPLE 1

Dimeric IgA is obtained by affinity purification from pooled healthy human plasma and conjugated with recombinant human secretory component forming secretory IgA. The secretory IgA is stabilized by the addition of human serum albumin to a final concentration of 5%. The final solution is adjusted to a therapeutic dose of 10 g secretory IgA daily. The secretory IgA is administered daily to a person suffering with intestinal lumen antibody deficiency. One week after initiation of treatment, the intestinal lumen antibody deficiency sufferer experiences diminution of his/her physiological abnormalities.

### EXAMPLE 2

The process of Example 1 is repeated with the secretory IgA administered enterically, at a higher daily dose of 20 g to achieve a similar result.

### REFERENCES

Agarwal S, Cunningham-Rundles C. Gastrointestinal Manifestations and Complications of Primary Immunodeficiency Disorders. Immunol Allergy Clin North Am. 2019 February:39(1):81-94. doi: 10.1016/j.iac.2018.08.006. PMID: 30466774; PMCID: PMC6537874.

Berzofsky JA, Berkower IJ., Epstein SL, Monoclonal Antibodies in Chapter 12, Antigen-Antibody Interactions and Monoclonal Antibodies, pp. 455-465 in Fundamental Immunology, Third Edition, W.E. Paul (ed), Raven Press, NY 1993. Berzofsky et al., Fundamental Immunology, Third Edition, 1993, pp 455-462.

Caubet JC, Nowak-Węgrzyn A. Current understanding of the immune mechanisms of food protein-induced enterocolitis syndrome. Expert Rev Clin Immunol. 2011 May;7(3):317-27.

Cohn EJ, Strong LE, Hughes WL, Jr., Mulford DJ, Ashworth JN, Melin M, Taylor HL, Preparation and Properties of Serum and Plasma Proteins IV. A System for the Separation into Fractions of the Protein and Lipoprotein Components of Biological Tissues and Fluids, J. Am. Chem. Soc. 1946; 68;459-475.

Corthesy B., Recombinant Secretory IgA for Immune Intervention Against Mucosal

Pathogens, Biochem. Soc. Trans. 1997, 25;471-475.

Crottet P, Cottet S, Corthesy B. Expression, Purification and Biochemical Characterization of Recombinant Murine Secretory Component, A Novel Tool in Mucosal Immunology, Biochem. J. 1999, 341;299-306.

Delacroix DL, Hodgson HJ, McPherson A, Dive C, Vaerman JP. Selective transport of polymeric immunoglobulin A in bile. Quantitative relationships of monomeric and polymeric immunoglobulin A, immunoglobulin M, and other proteins in serum, bile, and saliva. J. Clin. Invest. 1982 August;70(2):230-41.

Delacroix DL, Elkom KB, Geubel AP, Hodgson HF, Dive C, Vaerman JP. Changes in size, subclass, and metabolic properties of serum immunoglobulin A in liver diseases and in other diseases with high serum immunoglobulin A. J. Clin. Invest. 1983 February;71(2):358-67.

Deng M, Lv X, Liu L, Li J, Du G, Chen J, and Liu Y. Efficient Bioproduction of Human Milk Alpha-Lactalbumin in Komagataella phaffii J. Agric. Food Chem. 2022, 70, 8, 2664-2672.

Donaldson GP, Ladinsky MS, Yu KB, Sanders JG, Yoo BB, Chou WC, Conner ME, Earl AM, Knight R, Bjorkman PJ, Mazmanian SK Gut microbiota utilize immunoglobulin A for mucosal colonization. Science. 2018 May 18;360(6390):795-800.

Fadlallah J, El Kafsi H, Sterlin D, Juste C, Parizot C, Dorgham K, Autaa G, Gouas D, Almeida M, Lepage P, Pons N, Le Chatelier E, Levenez F, Kennedy S, Galleron N, de Barros JP, Malphettes M, Galicier L, Boutboul D, Mathian A, Miyara M, Oksenhendler E, Amoura Z, Doré J, Fieschi C, Ehrlich SD, Larsen M, Gorochov G. Microbial ecology perturbation in human IgA deficiency. Sci Transl Med. 2018 May 2;10(439).

Frossard CP, Hauser C, Eigenmann PA. Antigen-specific secretory IgA antibodies in the gut are decreased in a mouse model of food allergy. J Allergy Clin Immunol. 2004 August;114(2):377-82.

Granato DA, Piguet PF. A mouse monoclonal IgE antibody anti bovine milk beta-lactoglobulin allows studies of allergy in the gastrointestinal tract. Clin Exp Immunol. 1986 March;63(3):703-10.

Gopalakrishna KP, Macadangdang BR. Rogers MB, Tometich JT, Firek BA, Baker R, Ji J, Burr AHP, Ma C, Good M, Morowitz MJ, Hand TW. Maternal IgA protects against the development of necrotizing enterocolitis in preterm infants. Nat Med. 2019 July;25(7):1110-1115.

Harrison M, Kilby A, Walker-Smith JA, France NE, Wood CB. Cows' milk protein intolerance: a possible association with gastroenteritis, lactose intolerance, and IgA deficiency. Br Med J. 1976 Jun. 19;1(6024): 1501-4.

Harumi J. Non-IgE Mediated Food Allergy. Inflammation & Allergy-Drug Targets, 2008, 7, 3, 173-180(8).

Jones RML, Schweikart F, Frutiger S, Jaton J-C, Hughes GJ. Thiol-disulfide redox buffers maintain a structure of immunoglobulin A that is essential for optimal in vitro binding to secretory component. Biochimica et Biophysica Acta 1998; 1429:265-274.

Kabir S. Jacalin: a jackfruit (Artocarpus heterophyllus) seed-derived lectin of versatile applications in immunobiological research. J Immunol Methods 1998, 212:193-211.

Kelly CP, Chetham S, Keates S, Bostwick EF, Roush AM, Castagliuolo I, LaMont JT, Pothoulakis C. Survival of Anti-Clostridium difficile Bovine Immunoglobulin Concentrate in the Human Gastrointestinal Tract. Antimicrob Agents Chemother. 1997 February;41(2):236-41.

Khan R, Habbal M, Scaffidi MA, Bukhari AA, Rumman A, Al Ghamdi S, Betschel SD. Grover SC. Gastrointestinal Disease in Patients with Common Variable Immunodeficiency: A Retrospective Observational Study. J Can Assoc Gastroenterol. 2020 August;3(4):162-168. doi: 10.1093/jcag/gwz004. Epub 2019 Feb. 25. PMID: 32671325; PMCID: PMC7338846.

Kohler G, Milstein C. Continuous Cultures of Fused Cells Secreting Antibody of Predetermined Specificity, Nature 1975; 256;495-497.

Levy M, Kolodziejczyk AA, Thaiss CA, Elinav E. Dysbiosis and the immune system. Nat Rev Immunol. 2017 April;17(4):219-232.

Lullau E., Heyse S., Vogel H., Marison I., von Stockar U., Kraehanbuhl J-P., Corthesy B., Antigen Binding Properties of Purified Immunoglulin A Antibodies, J. Biol. Chem. 1996; 271:16300-16309.

Nakajima Al, Vogelzang A, Maruya M, Miyajima M, Murata M1, Son A, Kuwahara T, Tsuruyama T, Yamada S, Matsuura M5, Nakase H, Peterson DA, Fagarasan S, Suzuki K IgA regulates the composition and metabolic function of gut microbiota by promoting symbiosis between bacteria. J Exp Med. 2018 Aug. 6;215(8):2019-2034.

Oksenhendler E, Gérard L, Fieschi C. Malphettes M, Mouillot G, Jaussaud R. Viallard JF, Gardembas M, Galicier L, Schleinitz N, Suarez F, Soulas-Sprauel P, Hachulla E, Jaccard A, Gardeur A. Théodorou I, Rabian C, Debré P; DEFI Study Group. Infections in 252 patients with common variable immunodeficiency. Clin Infect Dis. 2008 May 15;46(10): 1547-54. doi: 10.1086/587669. PMID: 18419489.

Oncley JL, Melin M, Richert DA, Cameron JW, Gross PM, Jr., The Separation of the Antibodies, Isoagglutinins, Prothrombin, Plasminogen and β1-Lipoprotein into Subfractions of Human Plasma. J. Am. Chem. Soc. 1949; 71:541-550.

Possin ME, Morgan S, DaSilva DF, Tisler C, Pappas TE, Roberg KA, Anderson E, Evans MD, Gangnon R, Lemanske RF, Gern JE. The relationships among immunoglobulin levels, allergic sensitization, and viral respiratory illnesses in early childhood. Pediatr Allergy Immunol 2010: 21: 990-996.

Rogier, et al. Secretory antibodies in breast milk promote long-term intestinal homeostasis by regulating the gut microbiota and host gene expression (PNAS 2014 |111 (8) 3074-3079.

Schwarze J, Cieslewicz G, Joetham A, L Sun LK, Sun WN, Chang TW, Hamelmann E, W. Gelfand EW. Antigen-specific Immunoglobulin-A Prevents Increased Airway Responsiveness and Lung Eosinophilia after Airway Challenge in Sensitized Mice. Am J Resp Crit Care Med 1998; 158:519-525.

Shimoda M, Inoue Y, Azuma N, Kanno C. Local antibody response in Peyer's patches to the orally administered dietary protein antigen. Biosci Biotechnol Biochem. 1999 Dec;63(12):2123-9.

Simon MR, et al., Polyclonal antibody therapies for Clostridium difficile infection. Antibodies 2014;3:272-288.

Strong LE, Blood Fractionation, pp. 576-602 in vol. 3, Kirk-Othmer Encyclopedia of Chemical Technology. Second Edition, H.F. Mark, J.J. McKetta, D.F. Othmer (eds), Interscience Publishers, NY 1963, pp. 576-602.

Symersky J, Novak J, McPherson DT, DeLucas L, Mestecky J. Expression of the recombinant human immunoglobulin J chain in Escherichia coli. Mol. Immunol. 2000; 37:133-140.

Trajkovski V, Ajdinski L, Spiroski M. Plasma concentration of immunoglobulin classes and subclasses in children with autism in the Republic of Macedonia: retrospective study. Croat Med J. 2004 December;45(6): 746-9.

Vighi G, Marcucci F, Sensi L, G. Di Cara G, Frati F. Allergy and the gastrointestinal system. Clinical and Experimental Immunology, 153 (Suppl. 1): 3-6, 2008.

Vojdani A. Detection of IgE, IgG, IgA and IgM antibodies against raw and processed food antigens. Nutr Metab (Lond). 2009 May 12;6:22.

Walker AM, Kemp AS, Hill DJ, Shelton MJ. Features of transient hypogammaglobulinaemia in infants screened for immunological abnormalities. Arch Dis Child. 1994 March; 70(3): 183-186.

Wang M, Takeda K, Shiraishi Y, Okamoto M, Dakhama A, Joetham A, Gelfand EW. Peanut-induced intestinal allergy is mediated through a mast cell-IgE-FcepsilonRI-IL-13 pathway. J Allergy Clin Immunol. 2010 August;126(2):306-16, 316.e1-12.

Weifenbach N, Schneckenburger AAC. Lötters S. Global Distribution of Common Variable Immunodeficiency (CVID) in the Light of the UNDP Human Development Index (HDI): A Preliminary Perspective of a Rare Disease. J Immunol Res. 2020 Sep. 1;2020:8416124. doi: 10.1155/2020/8416124. PMID: 32953893.

Yang P-C, Wang C-S, An Z-Y. A murine model of ulcerative colitis: induced with sinusitis-derived superantigen and food allergen. BMC Gastroenterology 2005, 5:6.

Yoon MY, Yoon SS. Disruption of the Gut Ecosystem by Antibiotics. Yonsei Medical Journal. 2018, 59 (1):4.

Patent applications and publications mentioned in the specification are indicative of the levels of those skilled in the art to which the invention pertains. These applications and publications are incorporated herein by reference to the same extent as if each individual application or publication was specifically and individually incorporated herein by reference.

The foregoing description is illustrative of particular embodiments of the invention but is not meant to be a limitation upon the practice thereof. The following claims, including all equivalents thereof, are intended to define the scope of the invention.

1. A process for inhibiting symptoms of intestinal lumen antibody deficiency in a subject suffering therefrom, the process comprising:
administering orally to the subject suffering from intestinal lumen antibody deficiency a purified polymeric secretory IgA comprising a recombinant human secretory component and human plasma derived IgA dimer and higher polymers; and
allowing sufficient time for said purified polymeric secretory IgA to inhibit symptoms of the intestinal lumen antibody deficiency in the subject.

2. The process of claim 1 further comprising microencapsulating said purified polymeric secretory IgA prior to said administration.

3. The process of claim 1 wherein said human plasma derived IgA dimer is stabilized by the addition of human serum albumin prior to, or with said administration.

4. The process of claim 1 wherein said purified polymeric secretory IgA is stabilized by delivery with an antiacid.

5. The process of claim 1 wherein said purified polymeric secretory IgA is manufactured by an industrial process.

6. The process of claim 1 wherein the subject is a human.

7. The process of claim 1 wherein the intestinal antibody deficiency is caused by selective IgA deficiency.

8. The process of claim 1 wherein the intestinal antibody deficiency is caused by common variable immunodeficiency disorder.

9. The process of claim 1 wherein the administration is prophylactic.

10. A process for correction of intestinal lumen antibody deficiency in a subject, the process comprising:
administering orally to the subject a purified polymeric secretory IgA comprising a recombinant human secretory component and human plasma derived IgA dimer and higher polymers; and
allowing sufficient time for said purified polymeric secretory IgA to inhibit symptoms of the intestinal lumen antibody deficiency in the subject.

11. The process of claim 10 further comprising precluding further symptoms of the intestinal lumen antibody deficiency.

12. The process of claim 10 further comprising microencapsulating said purified polymeric secretory IgA prior to said administration.

13. The process of claim 10 wherein said human plasma derived IgA dimer is stabilized by the addition of human serum albumin prior to, or with said administration.

14. The process of claim 10 wherein said purified polymeric secretory IgA is stabilized by delivery with an antiacid.

15. The process of claim 10 wherein said purified polymeric secretory IgA is manufactured by an industrial process.

16. The process of claim 10 wherein the subject is a human.
